(19) United States
(12) Patent Application Publication
(10) Pub. No.: US 2023/0041342 A1
(43) Pub. Date: Feb. 9, 2023

(54) ALKYLATED NUCLEOSIDES, AND COMPOSITIONS AND METHODS THEREOF FOR NUCLEIC ACID DELIVERY

(71) Applicant: MICROVASCULAR THERAPEUTICS, LLC, Tucson, AZ (US)

(72) Inventors: Evan C. Unger, Tucson, AZ (US); Emmanuelle Joelle Meuillet, Tucson, AZ (US); Maria F. Acosta, Tucson, AZ (US); Dillon Hanrahan, Tucson, AZ (US)

(21) Appl. No.: 17/790,697

(22) PCT Filed: Jan. 7, 2021
(86) PCT No.: PCT/US21/12412

§ 371 Date: Jul. 1, 2022

Related U.S. Application Data
(60) Provisional application No. 62/958,328, filed on Jan. 8, 2020.

(51) Int. Cl. A61K 9/107; A61K 47/68; A61K 47/54; C12N 15/11

(52) U.S. Cl. CPC A61K 9/1075; A61K 47/6843; A61K 47/549; C12N 15/111; C12N 2320/32; C12N 2310/3513

(57) ABSTRACT

The invention provides novel compounds, compositions and formulations of liposomes, microbubbles and/or nanodroplets, and emulsions thereof, that are useful in delivery of various nucleic acids and genes (e.g., single stranded RNA, DNA, si-RNA and CRISPR constructs), as well as methods of preparation and use thereof including methods of imaging and gene delivery using ultrasound activation.

### PRIORITY CLAIMS AND RELATED PATENT APPLICATIONS

This application claims the benefit of priority from U.S. Provisional Application Ser. No. 62/958,328, filed on Jan. 8, 2020, the entire content of which is incorporated herein by reference for all purposes.

### TECHNICAL FIELDS OF THE INVENTION

This invention relates to compounds and pharmaceutical compositions and methods of their preparation and diagnostic or therapeutic use. More particularly, the invention relates to novel compounds, compositions and formulations of liposomes, microbubbles and/or nanodroplets, and emulsions thereof, that are useful in delivery of various nucleic acids and genes (e.g., single stranded RNA, DNA, si-RNA and CRISPR constructs), as well as methods of preparation and use thereof including methods of imaging and gene delivery using ultrasound activation.

### BACKGROUND OF THE INVENTION

Gene therapy is an emerging medical field which focuses on the utilization of the therapeutic delivery of nucleic acids into a patient's cells as a drug to treat or prevent diseases. Gene therapies include oligonucleotide-based drugs such as DNA, RNA, CRISPR and combinations thereof. An area of current great interest is CRISPR (clustered regularly interspaced short palindromic repeats), e.g., A CRISPR-Cas9, CRISPR-associated protein 9 in which RNA binds to the Cas9 enzyme. In CRISPR-Cas9, the modified RNA is used to recognize the DNA sequence, and the Cas9 enzyme cuts the DNA at the targeted location. Double stranded RNA may be used as si-RNA which may be used as catalytic RNA to prevent expression of a target gene. Several approved products are currently available based upon anti-sense oligonucleotides (ASO's). ASO's generally comprise constructs of single-stranded RNA which may, depending upon the target, block or accentuate gene expression and protein translation. All of the ASO's approved to date employ local administration.

There remains an ongoing need for novel and improved delivery technologies that enable systemic and/or localized delivery of a variety of gene-based therapeutics, including ASO's.

### SUMMARY OF THE INVENTION

Alkylated compounds comprising one or more nucleoside analogues (including both deoxyribonucleosides and ribonucleosides) are described which enable production of micelles, liposomes, nanoparticles, microspheres, emulsions, fluorocarbon emulsions and microbubbles. The alkylated nucleosides disclosed herein bind the correspond complementary nucleoside on the genetic material (“payload”) thereby incorporating the genetic material into the corresponding structure. The alkylated nucleosides (“carrier”), which are preferably charge neutrual, stabilize the genetic material and preserves it until the carrier delivers the payload to the target cells. Furthermore, the invention optionally comprises one or more targeting ligands to aid delivery to the selected desired cells. Optionally, ultrasound, or other energy source is used to monitor delivery of the genetic payload and to “activate” the carrier at the target site to release the genetic payload. By activation we refer to energy-mediated interaction with the carrier facilitating release, cellular and sub-cellular delivery of the genetic payload.

In one aspect, the invention generally relates to a compound comprising one or more nucleosides, or a derivative or analog thereof, covalently linked to one or more alkyl groups each having at least 9 (e.g., at least 12, at least 18) carbon atomes, or a pharmaceutically acceptable form thereof.

In certain embodiments, the one or more nucleosides, or a derivative or analog thereof, is covalently linked to the one or more alkyl groups via a linking group comprising a diphosphate moiety.

In certain embodiments, the one or more nucleosides, or a derivative or analog thereof, comprises one or more moieties selected from cytosine, adenine, guanine, uracil and thymine. In certain embodiments, the one or more nucleosides, or a derivative or analog thereof, comprises two or more moieties selected from cytosine, adenine, guanine, uracil and thymine. In certain embodiments, the one or more nucleosides, or a derivative or analog thereof, comprises four moieties selected from cytosine, adenine, guanine, uracil and thymine.

In certain embodiments, the one or more nucleosides, or a derivative or analog thereof, comprises one or more moieties selected from cytidine, adenosine, 5-methyluridine, uridine and guanosine.

In certain embodiments, the one or more nucleosides, or a derivative or analog thereof, comprises two or more moieties selected from cytidine, adenosine, 5-methyluridine, uridine and guanosine.

In certain embodiments, the one or more nucleosides are charge neutral nucleosides.

In certain embodiments, the one or more alkyl groups each has about 12 to about 24 (e.g., 12-16, 16-18, 18-24) carbon atoms. In certain embodiments, the compound has two alkyl groups each having about 12 to about 24 carbon atoms.

In certain embodiments, the one or more nucleosides comprise deoxyribonucleic acids. In certain embodiments, the one or more nucleosides comprise ribonucleic acids.

In certain embodiments, the compound further comprises a targeting ligand.

In another aspect, the invention generally relates to a complex comprising a compound disclosed herein, which is non-covalently complexed to a nucleic acid molecule. In certain embodiments, the nucleic acid molecule comprises a gene, an RNA or a CRISPR sequence.

In yet another aspect, the invention generally relates to a composition comprising a compound disclosed herein or a complex thereof conjugated to a nucleic acid.

In yet another aspect, the invention generally relates to micelles or liposomes comprising a compound disclosed herein or a complex thereof conjugated to a nucleic acid.

In yet another aspect, the invention generally relates to microbubbles comprising a compound disclosed herein or a complex thereof conjugated to a nucleic acid.

In yet another aspect, the invention generally relates to nanodroplets comprising a compound disclosed herein or a complex thereof conjugated to a nucleic acid.

In yet another aspect, the invention generally relates to a composition comprising micelles or liposomes disclosed herein.

In yet another aspect, the invention generally relates to a composition comprising microbubbles disclosed herein.

In yet another aspect, the invention generally relates to a composition comprising nanodroplets disclosed herein.

In certain embodiments, a fluorocarbon is employed to form microbubbles or nanodroplets. In certain embodiments, the fluorocarbon is selected from perfluoropropane, perfluorobutane and perfluoropentane.

In yet another aspect, the invention generally relates to a pharmaceutical composition comprising a compound disclosed herein or a complex thereof conjugated to a nucleic acid, or micelles, liposomes, microbubbles or nanodroplets comprising such compounds or complexes, and a pharmaceutically acceptable excipient, carrier, or diluent.

In yet another aspect, the invention generally relates to a method for treating a disease or condition comprising administering to a subject in need thereof a pharmaceutical composition comprising a compound disclosed herein or a complex thereof conjugated to a nucleic acid, or micelles, liposomes, microbubbles or nanodroplets comprising such compounds or complexes, and a pharmaceutically acceptable excipient, carrier, or diluent.

In certain embodiments, the disease or condition is selected from eye disease (uveitis, retinitis and retinal dystrophy), vascular and heart disease, cancer (acute lymphoblastic leukemia, B-cell lymphoma, head and neck squamous cell carcinoma and a wide variety of neoplastic conditions), pulmonary disease, Alzheimer's disease and other neurodegenerative conditions and lipoprotein lipase deficiency.

In yet another aspect, the invention generally relates to a method for delivering a nucleic acid to a target side, comprising administering to a subject a composition comprising a compound disclosed herein or a complex thereof conjugated to a nucleic acid, or micelles, liposomes, microbubbles or nanodroplets comprising such compounds or complexes, and a pharmaceutically acceptable excipient, carrier, or diluent.

### BRIEF DESCRIPTION OF THE DRAWINGS

The invention will be better understood from a reading of the following detailed description taken in conjunction with the drawings in which like reference designators are used to design like elements.

FIG. 1 shows a diagram of the chemical structure of dipalmitoylphosphatidyl-cytidine (phosphatidylcytidine).

FIG. 2 shows diagrams of the ribose nucleosides useful in preparation of alkylated nucleoside moieties.

FIG. 3A shows the binding of fluorescent poly-guanosine (poly-G) to the lipid 16:0 CDP DP. A 96-well plate was coated with the 16:0 CDP DP lipid and let dry overnight. Increasing concentrations of the poly-G (fluorescent) DNA sequence was added to the plate and was incubated for one hour to see if the sequence was bound to the lipid. A control lipid (DPPC) was used to see if the sequence was binding or not. The fluorescence intensity was measured using a plate reader after 1 hour of incubation at Ex=488 nm, Em=525 nm.

FIG. 3B shows a graph of binding of different concentrations of fluorescent poly-guanosine (poly-G) to microbubbles containing phosphatidylcytidine.

FIG. 4A shows the amount of fluorescent poly-G bound onto the microbubbles containing the lipid 16:0 CDP DP (1,2-dipalmitoyl-sn-glycero-3-(cytidine diphosphate) (ammonium salt)). Microbubbles formulated with CDP DP lipid were activated and then were incubated with the same dilutions from the previous assay four one hour. To get rid of unbound fluorescence sequence, the MBs were washed three times. Part of the MBs were plated in a 96-well plate (FIG. 4A) and the fluorescence intensity was measured using a plate reader after 1 hour of incubation at Ex=488 nm, Em=525 nm. The other part was observed under fluorescence microscope (FIG. 4B).

FIG. 4B shows a photomicrograph of microbubbles containing phosphatidylcytidine binding fluorescent poly-G.

FIG. 5 shows a photomicrograph of a cell wherein microbubbles containing phosphatidylcytidine binding poly-G are shown in and on the target cell.

FIG. 6 shows a synthetic scheme for production of di-alkyl-diphosphate nucleoside moieties. Conjugation of the nucleoside diphosphates to the free —OH of the diacylglycerol proceeds via dicyclohexylcarbodiimide (DCC)-mediated coupling, in the presence of a tertiary amine base, to afford the di-alkyl-diphosphate nucleotide products. Purification of the crude material is achieved via silica-gel chromatography.

FIG. 7A shows a synthetic scheme for production of neutral dialkyl-nucleoside moieties. Conjugations of both the diacylglycerol and the nucleoside moieties to a propionic acid-PEG4-propionic acid linker proceed under DCC-mediated coupling in the presence of a tertiary amine base. The symmetry of the linker allows either the diacylglycerol-linker or nucleoside-linker product to be produced and isolated independently for subsequent conjugation to the appropriate moiety, or potential one-pot synthesis of the dialkyl-nucleoside under proper stoichiometric control. Propionic Acid-PEG4-Propionic acid (50 mg) was dissolved in 1 mL diethyl ether and 55 μL SOCl₂ (5 eq) was added and stirred for 40 minutes. Pyridine was added (1.5 eq) along with 84 mg of 1,2-dipalmitoyl glycerol (1 eq) dissolved in 2 mL ether. Once fuming subsided, 50 mg of guanosine (1 eq) dissolved in 1 mL DMSO was added and the reaction stirred for one hour. The reaction was diluted with 5 mL each DMSO and ether, then with 5 mL water to remove unreacted thionyl chloride. The aqueous layer was separated and washed with ethyl acetate, and both organic layers were combined and washed with water. The solvent was evaporated to provide 58.7 mg of white powder (34% yield).

FIG. 7B. shows the mass spectrometry graph representing the nucleoside product as obtained using the chemical scheme in FIG. 7A.

FIG. 8 shows a synthetic scheme to prevent side reactions in the ribose moieties. In both synthetic procedures for nucleoside conjugation there is potential for undesired reactions with the free hydroxyls of ribose and amines of the nucleobase. Protection of the carbohydrate moiety with 2,2-dimethoxypropane (acetone dimethylacetal) affords the major 2,3-isopropylidene product with a free 5-OH for subsequent conjugation steps. This protection strategy is likely to afford imine derivatives of the nucleobase amines, also hindering side-reactivity on those sites. Conjugation of the protected moieties can proceed as described, followed by hydrolysis of the protecting groups under mildly acidic aqueous conditions to afford the products.

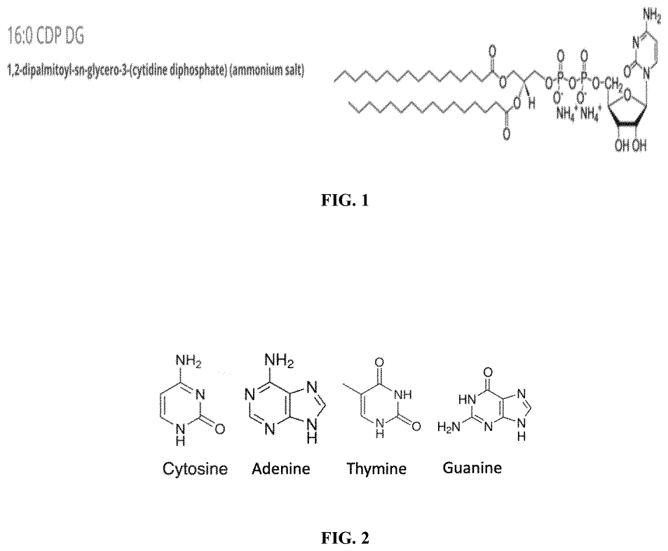

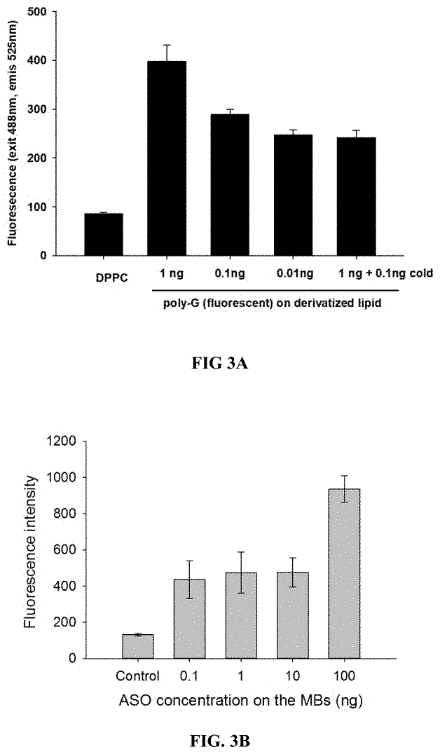

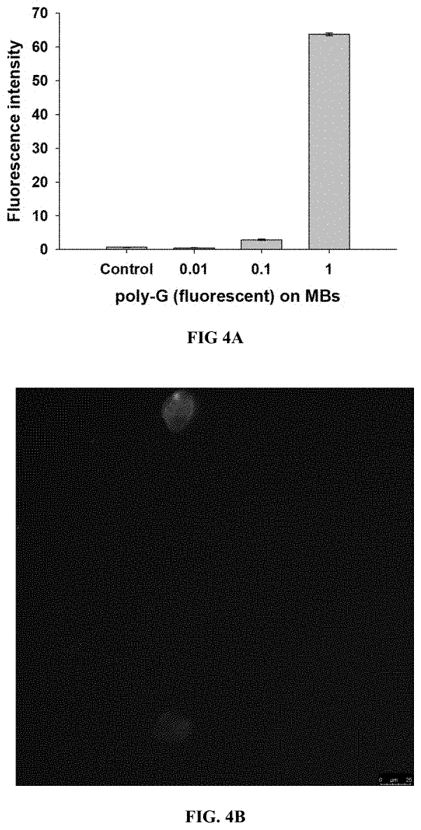

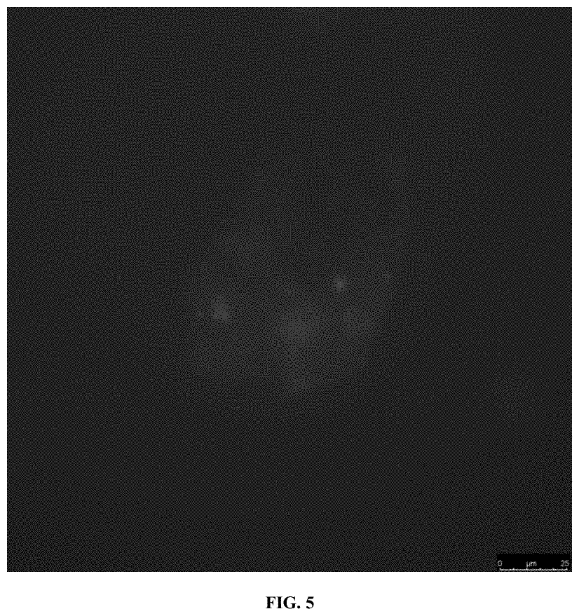

### DETAILED DESCRIPTION OF THE INVENTION

This invention is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment. The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention. In certain embodiments, Applicant's comprises one or more alkylated nucleoside analogues in admixture with one or more genetic constructs.

In one embodiment the alkylated nucleoside materials are formulated either by themselves or with one or more other alkylated moieties. Preferred alkylated moieties include fatty acids, cholesterol and its derivatives, phospholipids and fluorosurfactants. In general, one or more alkylated nucleoside analogues are formulated with the genetic construct to form a corresponding structure, generally ranging in size from the nanoscopic domain to microscopic size, e.g. from about 5 nm to about 5 microns in size. In one embodiment, fluorinated materials are incorporated into the composition comprising the alkylated nucleoside moieties with the genetic material to form emulsions, nanodroplets (ND) and microbubbles (MB). Herein, an emulsion may refer to a liquid in liquid structure that generally remains as such after administration to the subject. ND may refer to a material which is liquid but that may convert to a gaseous or other state upon changes of temperature or upon activation with energy such as light, magnetic, electrical energy or ultrasound energy. MB refers to gas within the construct. Preferred gases include fluorocarbon gases.

In one embodiment of the invention the nucleoside analogues comprise a monoalkyl group, e.g. a fatty acid attached to the nucleoside. In another embodiment they comprised cholesterol nucleoside analogues. In another embodiment, the invention comprises fluoro-alkyl moieties affixed to a nucleoside. In a preferred embodiment, the invention comprises di-alkyl moieties bound to the nucleoside headgroups.

The alkylated nucleosides are optionally formulated with one or more additional lipids. In certain embodiments, Applicant's phospholipid composition comprises one or more substantially charge-neutral phospholipids. In certain embodiments, Applicant's phospholipid composition comprises dipalmitoylphosphatidylcholine (“DPPC”). DPPC is a zwitterionic compound and is a substantially neutral phospholipid. In certain embodiments, Applicant's phospholipid composition comprises a second phospholipid comprising a polyhydroxy head group, and/or a headgroup of greater than 350 Daltons, wherein M is selected from the group consisting of Na⁺, K⁺, Li⁺, and NH₄⁺. A phospholipid may comprise an ammonium counterion and a polyethylene glycol (“PEG”) headgroup bound to the phosphoryl moiety. In certain embodiments, Applicant's composition comprises a PEG'ylated lipid. In certain embodiments, the PEG group MW is from about 1,000 to about 10,000 Daltons. In certain embodiments, the PEG group MW is from about 2,000 to about 5,000 Daltons. In certain embodiments, the PEG group MW is about 5,000 Daltons. In certain embodiments, Applicant's lipid composition includes one or more of the following PEG'ylated lipids: 1,2-dimyristoyl-sn-glycero-3-phosphoethanolamine-N-[methoxy(polyethylene glycol)-1000] (ammonium salt), 1,2-dipalmitoyl-sn-glycero-3-phosphoethanolamine-N-[methoxy(polyethylene glycol)-1000] (ammonium salt), 1,2-distearoyl-sn-glycero-3-phosphoethanolamine-N-1,2-dipalmitoyl-sn-glycero-3-phosphoethanolamine-N-[methoxy(polyethylene glycol)-3000] (ammonium salt), 1,2-distearoyl-sn-glycero-3-phosphoethanolamine-N-[methoxy(polyethylene glycol)-3000] (ammonium salt), 1,2-dioleoyl-sn-glycero-3-phosphoethanolamine-N-[methoxy(polyethylene glycol)-3000] (ammonium salt), 1,2-dimyristoyl-sn-glycero-3-phosphoethanolamine-N-[methoxy(polyethylene glycol)-5000] (ammonium salt), 1,2-dipalmitoyl-sn-glycero-3-phosphoethanolamine-N-[methoxy(polyethylene glycol) 5000] (ammonium salt), 1,2-distearoyl-sn-glycero-3-phosphoethanolamine-N-[methoxy(polyethylene glycol)-5000] (ammonium salt) and 1,2-dioleoyl-sn-glycero phosphoethanolamine-N-[methoxy(polyethylene glycol)-5000] (ammonium salt), 1,2-dimyristoyl-sn-glycero-3-phosphoethanolamine-N-[methoxy(polyethylene glycol)-2000] (ammonium salt), 1,2-dipalmitoyl-sn-glycero-3-phosphoethanolamine-N-[methoxy(polyethylene glycol)-2000] (ammonium salt), 1,2-distearoyl-sn-glycero phosphoethanolamine-N-[methoxy(polyethylene glycol)-2000] (ammonium salt), 1,2-dioleoyl-sn-glycero-3-phosphoethanolamine-N-1,2-dipalmitoyl-sn-glycero-3-phosphoethanolamine-N-[methoxy(polyethylene glycol)-3000] (ammonium salt), 1,2-distearoyl-sn-glycero-3-phosphoethanolamine-N-[methoxy(polyethylene glycol)-3000] (ammonium salt), 1,2-dioleoyl-sn-glycero-3-phosphoethanolamine-N-[methoxy(polyethylene glycol)-3000] (ammonium salt), 1,2-dimyristoyl-sn-glycero-3-phosphoethanolamine-N-[methoxy(polyethylene glycol)-5000] (ammonium salt), 1,2-dipalmitoyl-sn-glycero-3-phosphoethanolamine-N-[methoxy(polyethylene glycol)-5000] (ammonium salt), 1,2-distearoyl-sn-glycero-3-phosphoethanolamine-N-[methoxy(polyethylene glycol)-5000] (ammonium salt) and 1,2-dioleoyl-sn-glycero-3-phosphoethanolamine-N-[methoxy(polyethylene glycol)-5000] (ammonium salt) Phospholipid 5, shown above, represents dipalmitoyl-phosphatidylethanolamine, or DPPE. PE, particularly DPPE is a preferred lipid in the invention, preferably in the formulation with the other lipids at concentration of between 5 and 20 mole %, most preferably 10 mole %. In certain embodiments Applicant's invention contains one or more cone shaped or hexagonal HII forming lipids. Cone-shaped useful in the invention include monogalactosyl-diacylglycerol (MGDG), monoglucosyldiacylglycerol (MGDG), diphosphatidylglycerol (DPG) also called cardiolipin, phosphatidylserine (PS), phosphatidylethanolamine (PE) and diacylglycerol. Phosphatidic acid (PA) is also a cone-shaped lipid but is not preferred due to its propensity to hydrolysis and potential to cause bioeffects. The most preferred cone-shaped phospholipid is phoshatidylethanolamine (PE).

Examples of potentially useful cone-shaped cationic lipids include but are not limited to 1,2-dioleoyl-3-trimethyl-ammonium-propane (chloride salt), 1,2-dioleoyl-3-trimethylammonium-propane (methyl sulfate salt), 1,2-dimyristoyl-3-trimethylammonium-propane (chloride salt), 1,2-dipalmitoyl-3-trimethylammonium-propane (chloride salt), 1,2-distearoyl-3-trimethylanimonium-propane (chloride salt), 1,2-dioleoyl-3-dimethylammonium-propane, 1,2-dimyristoyl-3-dimethylammonium-propane, 1,2-dipalmitoyl-3-dimethylammonium-propane, 1,2-distearoyl-3-dimethylammonium-propane, dimethyldioctadecylammonium (sodium or bromide salt), 1,2-di-O-octadecenyl-3-trimethylanimonium propane (chloride salt), O,O-di-O-octadecenyl-3-ta-trimethylammonioacetyl-diethanolamine. Additional cationic lipids which may be useful include N1-[2-((1S)-1-[(3-aminopropyl)amino]-4-[di(3aminopropyl)amino]butyl-carboxamido)ethyl]-3,4-di[oleyloxy]-benzamide, 1,2-di-O-octadecenyl-3-trimethyl-ammonium propane (chloride salt) (DOTMA), 1,2-dipalmitoyl-sn-glycero-3-ethylphosphocholine (chloride salt)(which may be utilized in varying chain lengths from 12-18 carbons, saturated, unsaturated or mixed chains, e.g. saturated with unsaturated), Dimethyldioctadecylammonium (Bromide Salt), N-(4-carboxybenzyl)-N,N-dimethyl-2,3-bis(oleoyloxy)propan-1-aminium (which may be utilized in varying chain lengths from 14-18 carbons, saturated, unsaturated or mixed chains, e.g. saturated with unsaturated), 1,2-dipalmitoyl-3-trimethylammonium-propane (chloride salt) (which may be utilized in varying chain lengths from 14-18 carbons, saturated, unsaturated or mixed chains, e.g. saturated with unsaturated), 3β-[N—(N′,N′-dimethylaminoethane)-carbamoyl]cholesterol hydrochloride, N⁴-Cholesteryl-Spermine HCl Salt and 1,2-dioleyloxy-3-dimethylaminopropane.

The cationic lipids may be employed to neutralize the charge of the RNA or DNA construct which is generally a polyanion. It is believed that the alkylated nucleosides will form base pairs with complimentary nucleosides in the RNA or DNA construct and that the base pairing will be a stronger attraction than the electrostatic interaction afforded by the cationic lipids. In this regard, when cationic lipids are employed this is done, not to bind the genetic material but to adjust the charge of the resulting nanostructure.

In general, when the alkylated nucleosides are used with lipids the concentration of alkylated nucleosides represents between about 1 and about 95 mole % or the total lipid in the formulation. More preferably the alkylated nucleosides range from about 5 to 50 mole % of the total lipid and still more preferably the alkylated nucleosides represent about 10 mole % of the total lipid in the formulation. As one skilled in the art would recognize the alkyl chains in the alkylated nucleoside moieties may be saturated or unsaturated and when di-alkyl nucleosides are used they may also be mixed systems, e.g., comprise both a saturated and unsaturated alkyl chain. Likewise, the lipids employed in the formulation in addition to the alkylated nucleosides may be saturated or unsaturated and when di-alkylated lipids are used (e.g. phosphocholine) these may also be mixed systems, e.g., comprising both saturated and unsaturated alkyl chains.

In one embodiment, the alkylated nucleoside analogues are incorporated into liposomes generally in a mole ratio of from about 5 mole % to about 50 mole %. A variety of lipids may be employed in this embodiment as known in the art.

In another embodiment, the nucleoside lipids are employed in emulsions and may comprise up to 100% of the lipids, but generally will be less than 90, 80 or preferably about 70-75% of the total lipids.

In other embodiments, the alkylated nucleoside analogues will be employed in microbubbles or nanodroplets as shown in the examples.

The invention produces a variety of different constructs useful for multiple different applications. The route of administration will depend upon the condition to be treated. The materials of this invention may be administered intravenously, by pulmonary administration (e.g. inhalation), orally, subcutaneously, transdermally, by inhalation, via nasal administration, intraperitoneally, vaginally, intra-cisternally and rectally.

The microbubbles and liposomes prepared with the nucleoside lipids are useful for treating pulmonary disease. Because the microbubbles are filled with gas, they have very low effective hydrodynamic diameter and favorable properties for lung delivery. The constructs can be administered into lung via inhalation. For inhalation, a nebulizer may be used. Useful nebulizers include Jet nebulizers which uses compressed gas to make an aerosol (tiny particles of medication in the air), Ultrasonic nebulizers which makes an aerosol through high-frequency vibrations and Mesh nebulizers where liquid passes through a very fine mesh to form the aerosol. Additionally, inhalers may be used to administer the products into the lung. Exemplary inhalers include hydrofluoroalkane inhalers or HFA inhalers (formerly referred to as metered dose inhalers or MDIs), dry powder inhalers (DPI) and soft mist inhalers (SMI). For use with dry powder inhalers the formulation may be provided as a dried powder.

One or more bifunctional PEG'ylated lipids may be employed. Bifunctional PEG′ylated lipids include but are not limited to DSPE-PEG(2000), Succinyl 1,2-distearoyl-sn-glycero-3-phosphoethanolamine-N-[succinyl(polyethylene glycol)-2000] (ammonium salt), DSPE-PEG(2000), PDP 1,2-distearoly-sn-glycero-3-phosphoethanolamine-N-[PDP (polyethylene glycol)-2000] (ammonium salt), DSPE-PEG (2000) Maleimide 1,2-distearoly-sn-glycero-3-phospho-ethanolamine-N-[maleimide(polyethyleneglycol)-2000] (ammonium salt), DSPE-PEG(2000) Biotin 1,2-distearoyl-sn-glycero-3-phosphoethanolamine-N-[maleimide(polyethylene glycol)-2000] (ammonium salt), DSPE-PEG(2000) Cyanur 1,2-distearoly-sn-glycero-3-phosphoethanolamine N-[cyanur(polyethylene glycol)-2000] (ammonium salt), DSPE-PEG(2000) Amine 1,2-distearoyl, -sn-glycero phosphoethanolamine-N-[amino(polyethylene glycol)-2000] (ammonium salt), DPPE-PEG(5,000)-maleimide, 1,2- distearoyl-sn-glycero-3-phosphoethanolamine-N-[dibenzocyclooctyl (polyethylene glycol)-2000] (ammonium salt), 1,2-distearoyl-sn-glycero phosphoethanolamine-N-[azido(polyethylene glycol)-2000] (ammonium salt), 1,2- distearoyl-sn-glycero-3-phosphoethanolamine-N-[succinyI (polyethylene glycol)-2000](ammonium salt), 1,2- distearoyl-sn-glycero-3-phosphoethanolamine-N-[carboxy (polyethylene glycol)-(ammonium salt), 1,2- distearoyl-sn-glycero-3-phosphoethanolamine-N-[maleimide(polyethylene glycol)-2000] (ammonium salt), 1,2-distearoyl-sn-glycero-3-phosphoethanolamine-N-[PDP (polyethylene glycol)-2000] (ammonium salt), 1,2-dis-tearoyl-sn-glycero-3-phosphoethanolamine-N4amino(poly-ethylene glycol)-2000] (ammonium salt), 1,2-distearoyl-sn-glycero-3-phosphoethanolamine-N-[biotiny!(polyethylene glycol)-2000] (ammonium salt), 1,2-distearoyl-sn-glycero-3-phosphoethanolamine-N-[cyanur(polyethylene glycol)-2000] (ammonium salt), 1,2-distearoyl-sn-glycero-3-phos-phosphoethanolamine-N-[folate(polyethylene glycol)-2000] (ammonium salt), 1,2-distearoyl-sn-glycero-3-phosphoethanolamine-N-[folate(polyethylene glycol)-5000] (ammonium salt), N-palmitoyl-sphingosine-1-{succinyl[methoxy(poly-ethylene glycol)2000 and N-palmitoyl-sphingosine-1-{succinyl[methoxy(polyethylene glycol)5000]}.

The bifunctional lipids may be used for attaching antibodies, peptides, vitamins, glycopeptides and other targeting ligands to the structures comprising the alkylated nucleosides. One or more targeting ligands may be incorporated into the corresponding structures. The PEG chains MW may vary from about 1,000 to about 10,000 Daltons. In certain embodiments, the PEG chains MW are from about 2,000 to about 5,000 Daltons.

The lipid chains of the lipids used in the invention may vary from about 12 to about 24 carbons in length. Most preferably the chain lengths are from about 16 to about 18 carbons. Chains may be saturated or unsaturated but are preferably saturated. Cholesterol and cholesterol derivatives may also be employed in the invention with the proviso that they be neutral, or if charged contain a head group greater than about 350 MW in juxtaposition to the charge to shield the charge from the biological milieu.

When bifunctional lipids are used to create targeting ligands, also referred to as bioconjugates, these targeting moieties are generally incorporated into the structures at about 0.25 to about 10 mole % of the total lipids, more preferably at about 0.5 to about 5 mole % and most preferably at about 1 mole % of the total lipid.

In various embodiments, the alkylated nucleosides are formulated as MB or ND. The core of the structures may comprise a gas or gaseous precursor. Representative gases and gaseous precursors include nitrogen, oxygen, sulfur hexafluoride, perfluoropropane, perfluorobutane, perfluoropentane, perfluorohexane or mixtures thereof. For the purposes of imaging and gene/ASO/si-RNA/CRISPR delivery, the ideal MB/gaseous precursor comprises core gases which have low aqueous solubility coupled with a boiling point below body temperature. This results in a MB/ND with a long circulation time, a long useful life span, and high echogenic qualities for visualization on ultrasound and for ultrasonic activation to facilitate gene delivery. Applicant's gaseous precursors include, for example, fluorinated carbons, perfluorocarbons, sulfur hexafluoride, perfluoro ethers and combinations thereof. As the stilled artisan will appreciate, a particular fluorinated compound, such as sulfur hexafluoride, a perfluorocarbon or a perfluoro ether, may exist in the liquid state when the compositions are first made, and are thus used as a gaseous precursor. Whether the fluorinated compound is a liquid generally depends on its liquid/gas phase transition temperature, or boiling point. For example, a preferred perfluorocarbon, perfluoropentane, has a liquid/gas phase transition temperature (boiling point) of 29.5° C. This means that perfluoropentane is generally a liquid at room temperature (about 25° C.) but may be converted to a gas within the human body, the normal temperature of which is about 37° C., which is above the transition temperature of perfluoropentane. Thus, under normal circumstances, perfluoropentane is a gaseous precursor. As known to one skilled in the art, the effective boiling point of a substance may be related to the pressure to which that substance is exposed. This relationship is exemplified by the ideal gas law: PV=nRT, where P is pressure, V is volume, n is moles of substance, R is the gas constant, and T is 5 temperature in ° K. The ideal gas law indicates that as pressure increases, the effective boiling point also increases. Conversely, as pressure decreases, the effective boiling point decreases. Fluorocarbons for use as gaseous precursors in the compositions of the present invention include partially or fully fluorinated carbons, preferably perfluorocarbons that are saturated, unsaturated or cyclic. The preferred perfluorocarbons include, for example, perfluoromethane, perfluoroethane, perfluoropropane, perfluorocyclopropane, perfluorobutane, perfluorocyclobutane, perfluoropentane, perfluorocylcopentane, perfluorohexane, perfluorocyclohexane, and mixtures thereof. More preferably, the perfluorocarbon is perfluorohexane, perfluoropentane, perfluoropropane or perfluorobutane.

Preferred ethers include partially or fully fluorinated ethers, preferably perfluorinated ethers having a boiling point of from about 36° C. to about 60° C. Fluorinated ethers are ethers in which one or more hydrogen atoms is replaced by a fluorine atom. Preferred perfluorinated ethers for use as gaseous precursors in the present invention include, for example, perfluorotetrahydropyran, perfluoromethyltetrahydrofuran, perfluorobutylmethyl ether (e.g., perfluoro t-butylmethyl ether, perfluoro isobutyl methyl ether, perfluoro-n-butyl-methyl-ether, perfluoropropylmethyl ether, perfluoro isopropyl ethyl ether, perfluoro n-propyl ethyl ether, perfluorocyclobutylmethy ether, perfluorocyclopropylethyl ether, perfluoropropylmethyl ether (e.g., perfluoro isopropyl methyl ether, perfluoro n-propyl methyl ether), perfluorodiethyl ether, perfluorocyclopropylmethyl ether, perfluoroethylethyl ether and perfluorodimethyl ether.

Other preferred perfluoroether analogues contain between 4 and 6 carbon atoms, and optionally contain one halide ion, preferably Br—. For example, compounds having the structure Cn Fy Hx OBr, where n is an integer of from 1 to about 40, y is an integer of from O to about 13, and x is an integer of from O to about 13, are useful as gaseous precursors. Other preferable fluorinated compounds for use as gaseous precursors in the present invention sulfurhexafluoride and octafluoropropane.

Mixtures of different types of compounds, such as mixtures of a fluorinated compound (e.g., a perfluorocarbon or a perfluoroether) and another type of gas such as nitrogen may be employed.

Generally, preferred gaseous precursors undergo phase transition to gas at a temperature up to about 57° C., preferably from about 20° C. to about 52° C., preferably from about 37° C., to about 50° C., more preferably from about 38° C. to about 48° C., even more preferably from about 38° C. to about 46° C., still even more preferably from about 38° C. to about 44° C., even still more preferably from about 38° C., to about 42° C. Most preferably, the gaseous precursors undergo a phase transition at a temperature of about less than 40° C. As will be recognized by one skilled in the art, the optimal phase transition temperature of a gaseous precursor for use in a particular application will depend upon considerations such as, for example, the particular patient being treated, the tissue being targeted, the nature of the physiological stress state (e.g., cancer, infection or inflammation) causing the increased temperature, the stabilizing material used, and/or the genetic agent to be delivered.

Additionally, one skilled in the art will recognize that the phase transition temperature of a compound may be affected by local conditions within the tissue, such as, for example, local pressure (for example, interstitial, interfacial, or other pressures in the region). By way of example, if the pressure within the tissues is higher than ambient pressure, this will be expected to raise the phase transition temperature. The extent of such effects may be estimated using standard gas law predictions, such as Charles' Law and Boyle's Law. As an approximation, compounds having a liquid-to-gas phase transition temperature between about 30° C. and about 50° C. can be expected to exhibit about a 1° C. increase in the phase transition temperature for every 25 mm Hg increase in pressure. For example, the liquid-to-gas phase transition temperature (boiling point) of perfluoropentane is 29.5° C. at a standard pressure of about 760 mm Hg, but the boiling point is about 30.5° C. at an interstitial pressure of 795 mm Hg.

In one embodiment of the invention, the alkylated nucleosides are incorporated into a lipid blend and agitated with a fluorocarbon gas to form MB. The resulting MB are then subjected to decreased temperature and increased pressure to condense the gaseous cores into ND. For this application the preferred gases are perfluoropropane, perfluorobutane and perfluoropentane. To form ND of perfluoropropane, for example, the microbubble suspension may be cooled to about −17° C. and then pressurized to about 50 PSI (e.g., by injecting nitrogen gas or air into the vial). The milky white suspension of MB then becomes translucent bluish in color as the ND are formed. Less severe temperatures and pressures are needed to form the ND from perfluorobutane MB and even less so for perfluoropentane MB. Upon IV administration, the ND remain condensed (due to LaPlace pressures) but are activated back into MB upon insonation with ultrasound. The acoustic pressure required for activation of the MB into ND is lowest for perfluoropropane, intermediate for perfluorobutane and highest for perfluoropentane ND. By mixing varying concentrations of these gases it is possible to adjust tune the constructs to a given acoustic pressure at which the ND will convert to MB. For biomedical imaging ultrasound applications, the power levels are limited to avoid bioeffects. Alkylated nucleosides comprising a core of perfluoropropane carrying a payload of genetic drug can be activated at safe acoustic pressures, e.g. a mechanical index of ultrasound less than about 1.0. The advantage of the ND is that they have a smaller diameter (e.g. nanometer size) versus micron size for the MB. For delivering a payload of genetic material it is desirable that the material pass into the intracellular space. The smaller particles are thought to be advantageous for cellular delivery. In this regard the targeting ligands are desired to bind the particles to the cellular targets. A targeting regime engendering intracellular delivery is favored. As an example, E-selectin targeted particles are internalized by the cells. By incorporating a second ligand such as a vitamin, e.g. folate, or transferrin intracellular delivery may be achieved. Once the particles are internalized, they may enter endosomes and be hydrolyzed. Ultrasonic activation, however, will release the contents of the particles (e.g. gene payload) from the endosomes into the intracellular milieu. The genetic payload may then enter the requisite intracellular space, e.g. the nucleus for CRISPR or the ribosomes for ASO, etc.

In one embodiment of the invention the alkylated nucleosides (optionally formulated with one or more other lipids) may comprise a high boiling point fluorocarbon material, e.g. perfluorodecalin, perfluorooctylbromide, perfluorotripropylamine and other such fluorocarbons as would be known to one skilled in the art. Note that when the therapeutic genetic material is added to the alkylated nucleosides, these structures may shift position to form “rafts.” In so doing the alkylated nucleosides may re-orient to form base pairs with the RNA or DNA in the construct to be delivered. In this regard, the fluorocarbons may serve as an interface lowering surface tension and favoring the thermodynamics to allow the alkylated nucleosides to shift position so as to provide the most effective base-pairing.

In another embodiment, the alkylated nucleosides and associated genetic drugs are provided as dried powders using spray drying and/or lyophilization. A variety of cryoprotectants and stabilizing agents as are well know in the art may be used therein including but without limitation, trehalose, formamide, dimethyl sulfoxide and mixtures of formamide with DMSO, propylene glycol, glycerol, ethylene glycol, threitol and 2-methyl-2,4-pentanediol. As one skilled in the art would recognize the above aforementioned cryoprotectants may be used separately or in mixtures or in association with other cryoprotectants as are known in the art. Optionally, by using solvents such as glycerol and propylene glycol, the invention may be provided in a form substantially free of water and rehydrated with water or saline prior to use.

The Applicant's invention is useful for delivering a variety of RNA and DNA-based therapeutics. Antisense oligonucleotides are small pieces of DNA or RNA that bind to specific molecules of RNA. This generally blocks the ability of RNA to make a specific protein. An antisense oligonucleotide (ASO) may be referred to as such because its base sequence is complementary to the gene's messenger RNA, which is referred to as the “sense” sequence (so that a sense segment of messenger RNA “5′-AAGGUC-3′” would be blocked by the antisense messenger RNA segment “3′-UUCCAG-5′”). Historically, unmodified phosphodiester RNA ASOs are degraded following IV administration prior to reaching their target. It is believed that the Applicant's invention will help to stabilize ASOs so that they can reach the desired target. Nonetheless, other DNA and RNA derivatives may be used in this invention including Morpholino oligomers, e.g. DNA or RNA bases attached to a backbone of methylenemoipholine rings linked through phosphorodiamidate groups. ASOs (modified or unmodified) can interfere with pre-mRNA by preventing the binding of small nuclear ribonucleoproteins complexes from binding at the borders of introns on a strand of pre-mRNA and through other mechanisms, blocking or ribosome, activity and through other mechanisms. Peptide nucleic acids (peptide nucleic acids backbone is composed of repeating N-(2-aminoethyl)-glycine units linked by peptide bonds may also be used in the invention, Locked nucleic acids, modified RNA nucleotides in which the ribose moiety is modified with an extra bridge connecting the 2′ oxygen and 4′ carbon may also be used in the invention. In locked nucleic acids the bridge locks the ribose in the 3′-endo conformation. Locked nucleic acids can be mixed with DNA or RNA residues in oligonucleotides to enhance the hybridization properties. RNA interference can also benefit from the Applicant's invention, in which two types of small ribonucleic acid molecules—microRNA and small interfering RNA (siRNA) are exploited for RNA interference, siRNA is generally double stranded and comprises a passenger strand and a guide strand. The passenger strand is degraded, and the guide strand is incorporated into the RNA-induced silencing complex. Because of base pairing in the Applicant's invention this affords the potential to use a guide strand without a passenger strand for RNA interference. Plasmids, circular constructs of double-stranded DNA, generally ranging in size from 1 to over 1,000 kilobase pairs may be also be used in the Applicant's invention. Optionally, the plasmid DNA may be heated or subjected to chemical means to cause the two strands to partially fall apart so that base pairing between Applicant's alkylated nucleosides and the DNA may be optimized. Also, CRISPR, e.g. CRISPR-Cas9, in which a single guide RNA of the system recognizes its target sequence in the genome, and the Cas9 nuclease acts as a pair of scissors to cleave the double strands of DNA, may be used in the invention. The guide RNA will form base pairs with the alkylated nucleosides of this invention and can be designed to release the guide RNA when the CRISPR-Cas9 complex enters the cell. Cationic lipids can be incorporated into the formulation with Applicant's alkylated nucleosides to improve binding of double stranded DNA, RNA and CRISPR constructs. Likewise, cell-penetrating peptides and nuclear localization motifs can be incorporated into the Applicant's invention.

As one skilled in the art would recognize, Applicant's invention may be used to treat a wide variety of diseases employing the corresponding genetic construct to effect therapy. Without limitation, the invention may be used to treat eye disease (uveitis, retinitis and retinal dystrophy), vascular and heart disease, cancer (acute lymphoblastic leukemia, B-cell lymphoma, head and neck squamous cell carcinoma and a wide variety of neoplastic conditions), pulmonary disease, Alzheimer's disease and other neurodegenerative conditions and lipoprotein lipase deficiency. The Applicant's invention may also be used ex vivo, for example, to introduce one or more genes or other genetic constructs into cells, e.g. CAR T cells for administration to a patient to treat disease. A potential example would be to use Applicant's invention to target CAR T cells to treat p53 positive cancer. The invention may be used as a preclinical discovery tool in vivo and in vitro studies.

The following examples are meant to be illustrative of the practice of the invention and not limiting in any way.

### EXAMPLES

### Example 1. Preparation of Phosphatidylcholine Coated Perfluoropropane Microbubbles (MB) Referred to as MVT-100

A blend of lipids containing dipalmitoylphosphatidylcholine (DPPC), dipalmitoylphosphatidylethanolamine (DPPE) and dipalmitoylphosphatidylethanolamine-polyeythyleneglycol-5,000 (DPPE-MPEG-5000) was prepared. The lipids, suspended in propylene glycol, were heated to 70±50 C until they dissolved. The lipid solution was then added to an aqueous solution containing sodium chloride, phosphate buffer and glycerol and allowed to mix completely by stirring. Each ml of the resultant lipid blend contained 0.75 mg total lipid (consisting of 0.400 mg DPPC, 0.046 mg DPPE, and 0.32 mg MPEG-5000-DPPE). Each ml of the lipid blend also contained 103.5 mg propylene glycol, 126.2 mg glycerin, 2.34 mg sodium phosphate monobasic monohydrate, 2.16 mg sodium phosphate dibasic heptahydrate, and 4.87 mg sodium chloride in Water for Injection. The pH was 6.2-6.8. The material was provided in sealed vials with a headspace containing octafluoropropane (OFP) gas (>80%) with the balance air.

The microbubbles produced by the MVT-100 formulation remain stable over time with respect to concentration and size distribution even while suspended in normal saline. This blend of lipids, referred to as MVT-100, was used as the base MB for preparation of ND and for binding ASOs. To bind ASOs varying mole ratios of nucleoside phospholipids, e.g. phosphatidylcytidine were added.

### Example 2. Preparation of Phosphatidylcytidine Containing MB and Loading with ASO

Fluorescent labeled poly-G (Alexa Fluor 488) was obtained from Integrated DNA Technologies (IDT) and added to the lipid mixture (0.75 mg/ml, 73.8 mole % DPPC, 9 mole % DPPE, 6.3 mole % DPPE-PEG5000 and 10 mole % 16:0 CDP DG (cytidine diphosphate)). The MB were prepared by agitation. 10 μl of MB were incubated with different dilutions of the fluorescent labeled poly-G and then incubated for one hour. After one hour of incubation, the unbound fluorescent poly-G was removed by centrifugation (1500 rpm for 3 minutes) in Eppendorf tubes. The bottom clear liquid was drawn out with a syringe and the top milky MB layer was resuspended in fresh PBS. This was done 3 times.

### Example 3. Binding of ASO to MB Both with and without Phosphatidylcytidine

An aliquot of the MB bound to the ASO were seeded in 96 black well plates and the fluorescence intensity was measured using a plate reader (Molecular Devices, SpectraMax M3) at Ex [symbol] =488 nm, Em [symbol] =525 nm. An aliquot of the MB bound to the ASO was seeded onto poly-d-lysine coated glass bottom dishes (Mat Tek, Ashland, Mass.) and let them attach to the surface. The fluorescent MB were observed under a microscope using a Leica DMI6000 multifunction motorized inverted microscope.

### Example 4. Preparation of Nanodroplets both with and without phosphatidylcytidine

Perfluoropropane (PFP) MB based upon MVT-100 with proprietary non-critical excipients were condensed with low temperature by incubating in a bath of ethyleneglycol at −17° C. and 50 PSI for 5 min. The MB appeared as a whitish foam, but after the condensation process, the ND appeared as a light bluish translucent emulsion. The resulting ND had mean particle size=600 nm and PFP concentration of 90% compared to parent MVT-100 MB which had mean particle size=830 nm and PFP concentration=90% in the headspace.

### Example 5. Incubation of Phosphatidylcytidine MB with Cells

An aliquot of the MB bound to the ASO was added to human epithelial colorectal adenocarcinoma cells (CaCo2). Cells were grown in T25 flasks using ATCC-formulated Eagle's Minimum Essential Medium (EMEM), supplemented with 20% of fetal bovine serum and 1% of penicillin-streptomycin. Cells were incubated in a 5% carbon dioxide, humidified atmosphere at 37° C. After confluence, cells were detached with trypsin and transferred onto poly-d-lysine coated glass bottom dishes, followed by incubation and additional 24 hours to ensure adherence. MB were added and incubated with the cells for 2 hours. After 2 hours cells were washed to remove the unbound fluorescent MB. Cells were observed under a microscope using a Leica DMI6000 multifunction motorized inverted microscope.

### Prophetic Example 1. Preparation of MB Containing Four Different Nucleoside Lipids

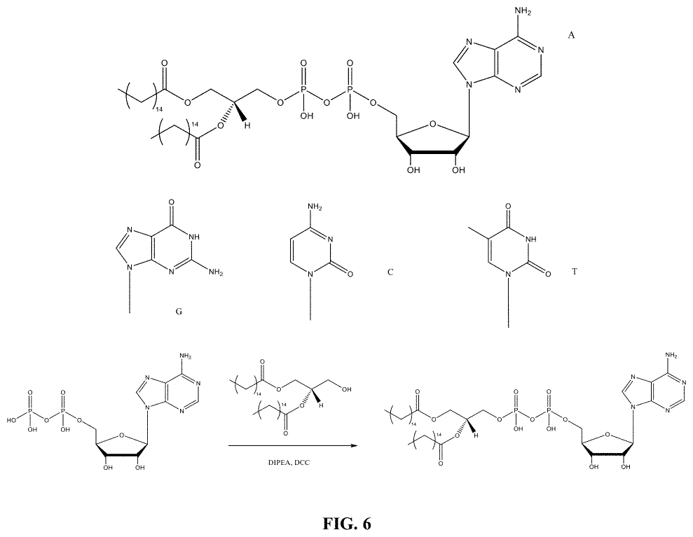

MB were prepared from the lipids described in Example 1, except that 10 mole % of the lipid was substituted with four different nucleoside lipids. Following the synthetic scheme shown in FIG. 6, phosphatidyl-cytidine, adenine, thymine and guanine were prepared. Each of the corresponding phosphatidyl nucleosides was purified by HPLC. Each phosphatidyl nucleoside moiety was added to the formulation at 2.5 mole % so that the aggregate mole % of total phosphatidyl nucleosides was at 10 mole %. The resulting MB were shown to avidly bind ASO. A serum stability assay showed that the MB containing phosphatidyl nucleosides provided significant improvement in stability compared to ASO without MB.

### Prophetic Example 2. Preparation of ND Containing Four Different Nucleoside Lipids

MB were prepared as in Prophetic Example 1. The resulting MB were exposed to decreased temperature and increased pressure as in Example 4. The ND were then incubated with ASO.

### Prophetic Example 3. Preparation of ND Targeted (tND) to E-Selectin for ASO Delivery

The bioconjugate of E-selectin binding peptide is prepared from DSPE-PEG-Maleimide and DK12-OH peptide to yield the resulting bioconjugate. HPLC is performed to purify the bioconjugate and mass spec is performed confirming the structure. To make tND, typically 1 mole % of the bioconjugate is mixed with 76 mole % DPPC and 7 mole % DPPE-MPEG(5000) and 7 mole % DPPE and 10 mole % of the nucleoside phosphatidylcholine lipids described in Prophetic Example 1. The lipids are dissolved in a diluent of buffered normal saline, propylene glycol and glycerol 76/7/7/10 molar ratios. The clear mixture of lipids is placed in sealed vials which are filled with octafluoropropane gas. ND formulations will be characterized for particle sizes and concentrations using an Accusizer™ 780 (Particle Sizing Systems, Port Richey, Fla.) and a NanoBrook 90 Plus (Brookhaven) respectively to ensure the uniformity of ND preparations. The concentration of perfluoropropane in the different formulations is measured by Raman spectroscopy (DXR2 Smart Raman, ThermoScientific). The ASOs for E-selectin and ICAM-1, as phosphorothioate analogues, are incubated with the NDs and unbound ASO is dialyzed away. The resulting NDs are targeted to E-selectin and are useful for decreasing inflammatory conditions such as uveitis, arthritis and other related conditions.

### Prophetic Example 4. Preparation of Liposomes Binding ASOs

The lipids described in Prophetic Example 4 are employed to product liposomes. For this application, no fluorocarbon gas is employed. After the lipids are rehydrated liposomes are produced by freeze-thaw followed by extrusion of the liposomes. ASOs are added to the liposomes and the unbound ASO is removed by dialysis. The resulting liposomes targeted to E-selectin are useful for delivering ASO to inflammatory conditions.

### Example 5. Preparation of Emulsions Useful for Binding ASOs

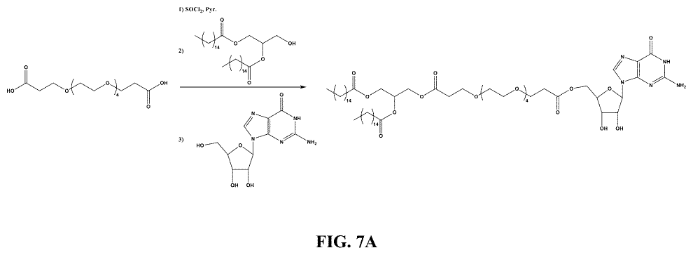

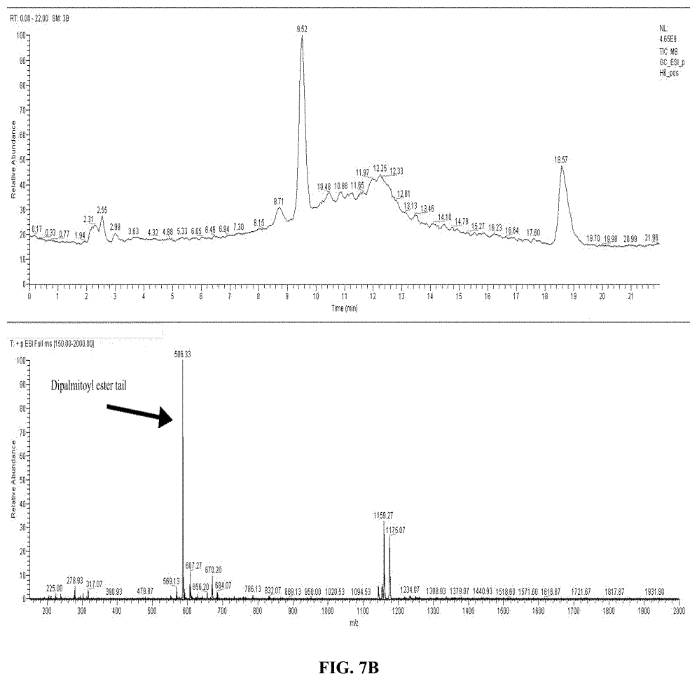

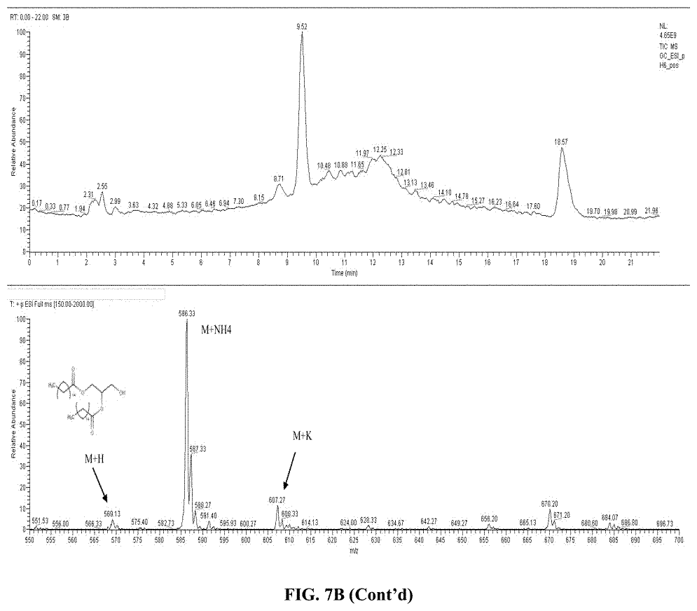

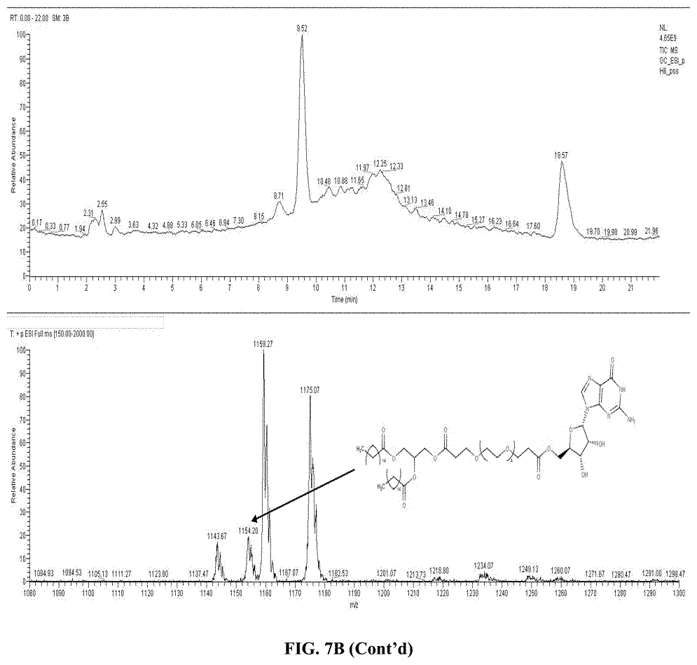

Neutral lipids of FIG. 7A-7B are prepared with cytidynil, adenine, thymine and guanine headgroups. The lipids are formulated without additional lipids and form micelles. ASOs are added to the resulting micelles to form complexes.

### Prophetic Example 6. Preparation of Nucleoside Lipids to Eliminate Ribose Side Reactions

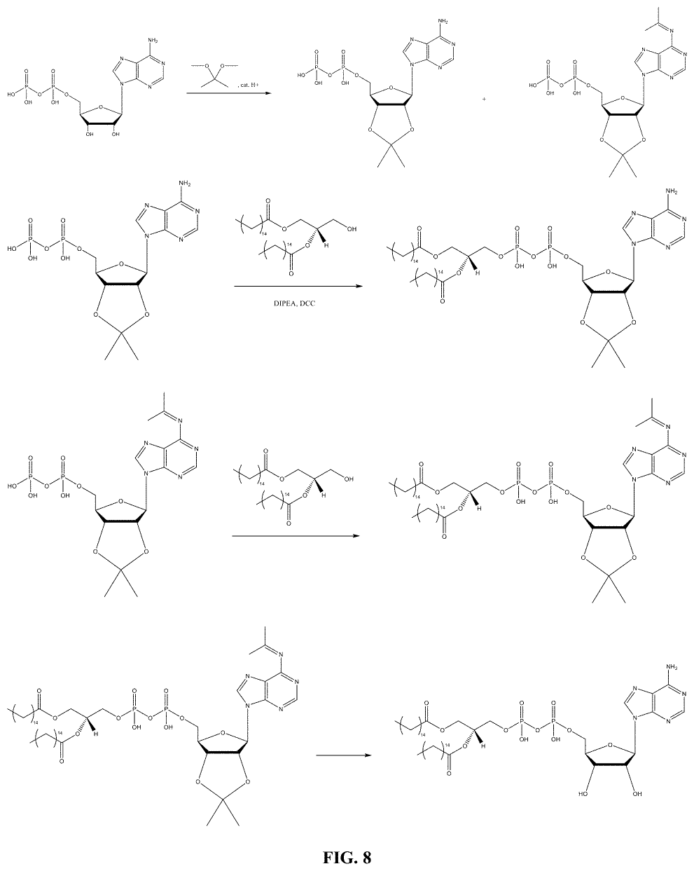

The nucleoside lipids shown in FIG. 8 are produced and purified by HPLC. The resulting nucleoside lipids are useful for binding ASOs.

### Prophetic Example 7. Imaging and Treatment of a Patient with Uveitis

ND are prepared as above with contain 10 mole % of nucleoside lipids with cytidynil, adenine, thymine and guanine headgroups. ASOs to E-selectin and ICAM-1 are added to the ND and bound thereto. The ND contain a trace amount of the fluorophore DiO. Approximately 2.5 ml of solution containing approximately 10¹⁰ ND and about 2 mg each of ASO to E-Selectin and ICAM-1 are injected IV into a patient with uveitis. Fundoscopy shows uptake of ND in the inflamed retina. Ultrasound imaging with a 20 MHz transducer shows uptake of ND into the inflamed regions of the eye. A 1.0 MHz transducer is then used at a power level of 720 milliwatts to cavitate the ND/MB, releasing the ASOs from the endosomes in the inflamed endothelial cells and macrophages. Follow-up imaging two weeks later using E-selectin targeted MB shows much less uptake reflecting decrease in inflammation.

### Prophetic Example 8. Lung Delivery and Treatment of Pulmonary Disease

The microbubbles and liposomes prepared with the nucleoside lipids are useful for treating pulmonary diseases.

A. The neutral nucleoside lipids are mixed with the lipids dipalmityolyphoshatidylcholine (DDPC), dipalmitoylyphosphatidylethanolamine (DPPE) and DPPE-PEG(5,000). The final concentrations of lipids are about 10-20 mole % nucleoside lipids and 80-90 mole % of DPPC, DPPE and DPPE-PEG. The ratios of the non-nucleoside lipids are about 82 mole % DPPC, 10 mole % DPPE and 8 mole % DPPE-PEG. The lipids are suspended in liquid comprising about 80 w/vol % normal saline with 10 v/vol % propylene glycol and 10 w/vol % glycerol. The total lipids=about 2 mg per ml and are placed in a glass Wheaton vial, 1.5 ml volume with a head space of perfluorobutane and the vials are sealed. The vials are shaken at a speed of about 4,500 RPM on an amalgamator device yielding microbubbles with about 10 to 20 mole percent nucleoside lipids. The microbubbles are then mixed with roughly 1:1 weight/vol of lipid with w/vol of antisense oligonucleotides targeting TGF-β mRNA and agitated gently and then administered via an ultrasonic nebulizer to a patient suffering from idiopathic pulmonary fibrosis. The patient inhales the nebulized microbubbles carrying the antisense oligonucleotides. Multiple treatments are administered resulting in amelioration of disease over a period of several months.

B. The above is substantially repeated except that the antisense oligonucleotides (ASO) are added to the aqueous suspension of lipids in the vials and the agitation step is repeated such that the microbubbles bind the ASOs as the microbubbles are formed.

C. The above of Example A is substantially repeated except that the lipids are dissolved in propylene glycol, heated to 55° C. with the ASOs and filtered through 0.2 micron filters. The material is then used to fill the vials and sealed with perfluorobutane gas. The resulting product is essentially anhydrous. The vials are shaken at about 4,500 RPM for 45 seconds and then the microbubbles are rehydrated by injecting about 80% w/vol saline with about 10% w/vol glycerol into the vials. The material is gently agitated withing the vials, withdrawn via syringe and placed into the ultrasonic nebulizer for administration to a patient.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Unless specifically stated or obvious from context, as used herein, the term “or” is understood to be inclusive.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A compound comprising one or more nucleosides, or a derivative or analog thereof, covalently linked to one or more alkyl groups each having at least 9 carbon atoms, or a pharmaceutically acceptable form thereof.

2. The compound of claim 1, wherein the one or more nucleosides, or a derivative or analog thereof, is covalently linked to the one or more alkyl groups via a linking group comprising a diphosphate moiety.

3. The compound of claim 2, wherein the one or more nucleosides, or a derivative or analog thereof, comprises one or more moieties selected from cytosine, adenine, guanine, uracil and thymine.

4. The compound of claim 3, wherein the one or more nucleosides, or a derivative or analog thereof, comprises two or more moieties selected from cytosine, adenine, guanine, uracil and thymine.

5. The compound of claim 2, wherein the one or more nucleosides, or a derivative or analog thereof, comprises one or more moieties selected from cytidine, adenosine, 5-methyluridine, uridine and guanosine.

6. The compound of claim 5, wherein the one or more nucleosides, or a derivative or analog thereof, comprises two or more moieties selected from cytidine, adenosine, 5-methyluridine, uridine and guanosine.

7. The compound of claim 1, wherein the one or more alkyl groups each having about 12 to about 24 carbon atoms.

8. The compound of claim 7, comprising two alkyl groups each having about 12 to about 24 carbon atoms.

9. The compound of claim 1, wherein the one or more nucleosides comprise deoxyribonucleic acids.

10. The compound of claim 1, wherein the one or more nucleosides comprise ribonucleic acids.

11. The compound of claim 1, wherein the one or more nucleosides is charge neutral.

12. The compound of claim 1, further comprising a targeting ligand.

13. The compound of claim 12, wherein the targeting ligand is selected from antibodies, peptides, vitamins and glycopeptides.

14. The compound of claim 13, wherein the targeting ligand is an antibody.

15. A complex comprising a compound of claim 1 non-covalently complexed to a nucleic acid molecule.

16. The complex of claim 15, wherein the nucleic acid molecule comprises a single stranded RNA molecule, a DNA molecule, an si-RNA molecule, a CRISPR construct, or an anti-sense oligonucleotide.

17. (canceled)

18. (canceled)

19. (canceled)

20. (canceled)

21. (canceled)

22. A micelle or liposome comprising a compound of claim 1.

23. (canceled)

24. A microdroplet or nanodroplet comprising a compound of claim 1.

25. (canceled)

26. (canceled)

27. (canceled)

28. (canceled)

29. (canceled)

30. A pharmaceutical composition comprising a compound of claim 1, and a pharmaceutically acceptable excipient, carrier, or diluent.

31. A method for treating a disease or condition comprising administering to a subject in need thereof a pharmaceutical composition comprising a compound of claim 1.

32. (canceled)

33. (canceled)

34. (canceled)

35. (canceled)

36. (canceled)

37. (canceled)
